(19) United States
(12) Patent Application Publication
(10) Pub. No.: US 2014/0039625 A1
(43) Pub. Date: Feb. 6, 2014

(54) A MINIMALLY-INVASIVE, LATEROVERTICALLY EXPANDING, INTERVERTEBRAL DISC SCAFFOLDING

(71) Applicants: JOHN J. FLYNN, Walnut Creek, CA (US); PAUL J. BIRKMEYER, Marshfield, MA (US); JOHN TO, Newark, CA (US); OUROBOROS MEDICAL, INC, Fremont, CA (US)

(72) Inventors: JOHN TO, NEWARK, CA (US); JOHN J. FLYNN, WALNUT CREEK, CA (US); PAUL J. BIRKMEYER, MARSHFIELD, MA (US)

(21) Appl. No.: 13/954,914

(22) Filed: Jul. 30, 2013

Related U.S. Application Data
(60) Provisional application No. 61/678,070, filed on Jul. 31, 2012.

(51) Int. Cl. A61F 2/44; A61F 2/46

(52) U.S. Cl. CPC A61F 2/4455; A61F 2/4601
USPC 623/17.16

(57) ABSTRACT

A laterovertically expandable scaffolding is provided for supporting an intervertebral disc space using a minimally invasive procedure. The scaffolding can be configured to provide a low-profile entry in a collapsed configuration through the single point of entry through the annulus. The expanding including laterally expanding at least a portion of a first support and at least a portion of a second support away from each other; and, vertically expanding at least a portion of the first support or at least a portion of the second support for a distraction of the intervertebral space. The lateral movement can include a rotation at a point of intersection between the first support and the second support, the intersection being biased anteriorly in the intevertebral space to facilitate the adding of the grafting material.

### CROSS-REFERENCE TO RELATED APPLICATIONS

This application claims the benefit of U.S. Provisional Application No. 61/678,070, filed Jul. 31, 2012, which is hereby incorporated herein by reference in its entirety.

### BACKGROUND

1. Field of the Invention

The teachings herein are directed to intervertebral scaffoldings and methods of creating the same.

2. Description of the Related Art

Intervertebral disc disease is a major worldwide health problem. In the United States alone almost 700,000 spine procedures are performed each year and the total cost of treatment of back pain exceeds $30 billion. Age related changes in the disc include diminished water content in the nucleus and increased collagen content by the 4.sup.th decade of life. Loss of water binding by the nucleus results in more compressive loading of the annulus. This renders the annulus more susceptible to delamination and damage. Damage to the annulus, in turn, accelerates disc degeneration and degeneration of surrounding tissues such as the facet joints.

The two most common spinal surgical procedures performed are discectomy and spinal fusion. These procedures only address the symptom of lower back pain, nerve compression, instability and deformity. Traditionally fusion cages such as the Medtronic CAPSTONE cage are oversized to the disc space to distract as it is inserted. However this makes it difficult to insert and position properly. Recently a number of new fusion cages such as the Globus CALIBER cage can be inserted at a low height and expanded vertically to distract the disc space. However, such cage have the typical limitation in that it is not symmetrical about the sagittal plane if it is loaded from one side in a common approach called the Transforaminal Lumbar Interbody Fusion (TLIF), it does not provide a path for bone graft to be insertion to fill in the space surrounding the cage, it does not conform to the nonplanar surface of the endplate, and it cannot expand laterally to increase the footprint relative to size of the insertion. The last limitation requires that it be inserted through a large opening through the body tissues to accommodate a large enough cage for stability, and this large opening necessitates more trauma for the patient. As such, the art would benefit from a device that can be used to (i) laterally expand within the native annulus, (ii) vertically expand for distraction of the intervertebral space, (iii) provide additional space around the device in the annulus for the introduction of graft materials; (iv) provide a large, symmetrical footprint to maximize uniform load distribution against the endplate; (v) conform to the non planar geometry of the endplate to maximize surface contact; and (vi) insert into the annulus in a minimally-invasive manner using only a unilateral approach.

### SUMMARY

The teachings provided herein are generally directed to a method of fusing an intervertebral space using a laterovertically-expandable scaffolding.

In some embodiments, the method comprises creating a single point of entry into an intervertebral disc, the intervertebral disc having a nucleus pulposus surrounded by an annulus fibrosis, and the single point of entry is created through the annulus fibrosis. The method includes removing the nucleus pulposus from within the intervertebral disc through the single point of entry, leaving an intervertebral space for expansion of a laterovertically-expandable scaffolding within the annulus fibrosis. The method further includes inserting the laterovertically-expandable scaffolding through the single point of entry into the intervertebral space, the laterovertically-expandable scaffolding having at least a first support and a second support, the combination of the first support and the second support operable to laterally expand and vertically expand from a collapsed configuration within an intervertebral space, such that the laterovertically-expandable scaffolding is configured to provide a low-profile entry in the collapsed configuration through the single point of entry through the annulus. As such, the method further includes expanding the laterovertically-expandable scaffolding. The expanding includes laterally expanding at least a portion of the second support and at least a portion of the first support away from each other; and, vertically expanding at least a portion of the first support or at least a portion of the second support for a distraction of the intervertebral space. As a method of fusing the intervertebral space, the method further includes adding a grafting material through the single point of entry into the intervertebral space around the laterovertically-expandable scaffolding. In such embodiments, the first support and the second support are each at least substantially rigid; and, the first support and the second support lie at least substantially on the same plane.

It should be appreciated that the single point of entry can be made in any manner that will facilitate obtaining the functions taught herein. In some embodiments, the single point of entry through the annulus fibrosis is configured to accommodate the low profile having an area having an effective diameter ranging from about 5 mm to about 12 mm.

The teachings are also directed to a laterovertically expandable scaffolding for fusing an intervertebral disc space. In some embodiments, the scaffolding comprises at least a first support and a second support, the combination of the first support and the second support operable to laterally expand and vertically expand from a collapsed configuration within an intervertebral space. In these embodiments, the first support and the second support can be at least substantially rigid; and, the first support and the second support can lie at least substantially on the same plane. The collapsed configuration can be configured to provide a low-profile entry through a minimally-invasive single point of entry through the annulus fibrosis of an intervertebral disc, the intervertebral disc having the nucleus pulposus removed. The removal of the nucleus pulposus leaves an intervertebral space for expansion of the laterovertically-expandable scaffolding within the annulus fibrosis using an expansion mechanism for laterally expanding at least a portion of the second support and at least a portion of the first support away from each other; and, vertically expanding at least a portion of the first support or at least a portion of the second support for a distraction of the intervertebral space.

The teachings are also directed to a laterally expandable scaffolding for fusing an intervertebral disc space. In these embodiments, the scaffolding comprises at least a first support and a second support, the combination of the first support and the second support operable to laterally expand and vertically expand from a collapsed configuration within an intervertebral space; and, an expansion mechanism. In these embodiments, the first support and the second support can be at least substantially rigid; and, the first support and the second support can lie at least substantially on the same plane. In these embodiments, the collapsed configuration can be configured to provide a low-profile entry through a minimally-invasive single point of entry through the annulus fibrosis of an intervertebral disc. The intervertebral disc has the nucleus pulposus removed, leaving an intervertebral space for expansion of the laterovertically-expandable scaffolding within the annulus fibrosis.

In some embodiments, the expansion mechanism can laterally expand at least a portion of the second support and at least a portion of the first support away from each other, the laterally expanding including a rotation at a point of intersection between the first support and the second support, such that the laterally expanding includes a scissor-like movement between the first support and the second support in the intervertebral space; and, the vertically expanding includes expanding at least a portion of the first support or at least a portion of the second support for a distraction of the intervertebral space, the vertically expanding includes introducing a vertical expansion member into the intervertebral space through the single point of entry and into the first support or the second support of the scaffolding to provide a vertical force on adjacent vertebral endplates for the distraction of the intervertebral space. In these embodiments, the collapsed configuration is configured for the low profile entry through the annulus fibrosis, having the shape of an I for inserting the scaffolding into the intervertebral space through the single point of entry; and, the expanded configuration is configured to provide a stable support for fusing the intervertebral space, having the shape of an X in the intervertebral space, the point of intersection biased anteriorly in the intervertebral space to facilitate the adding of a grafting material and maximize an area of contact between the scaffolding, the grafting material, and the vertebral endplates of the intervertebral space.

It should be appreciated that the collapsed configuration of the scaffolding can be any configuration that will facilitate obtaining the functions taught herein. In some embodiments, the collapsed configuration has the shape of an I for the inserting of the scaffolding into the intervertebral space, and the expanded configuration has the shape of an X in the intervertebral space. In some embodiments, the shape of the X is asymmetrical in the intervertebral space and the intersection is biased anteriorly in the intervertebral space to facilitate the adding of the grafting material and maximize an area of contact between the scaffolding, the grafting material, and the vertebral endplates of the intervertebral space. For example, the low profile entry of the scaffolding in the collapsed configuration contributes to the minimally-invasive nature of the treatments taught herein, and any low profile entry that accomplishes the reduction of trauma sought herein can be used. In some embodiments, the low profile entry has an area with an effective diameter ranging from about 5 mm to about 12 mm for a minimally-invasive single point of entry through the annulus fibrosis.

It should be appreciated that the lateral and vertical expansions can occur in any manner, using any respective expansion mechanism that will provide the functions of the scaffoldings taught herein. In some embodiments, the laterally expanding includes a rotation at a point of intersection between the first support and the second support, such that the laterally expanding includes a scissor-like movement between the first support and the second support in the intervertebral space. And, in some embodiments, the laterally expanding includes a translation at a point of intersection between the first support and the second support, such that the laterally expanding includes a scissor-like movement in the intervertebral space between the first support and the second support. In some embodiments, the vertically expanding includes expanding the first support or the second support using a means for creating a convex surface that at least substantially complements the concavity of a surface of a vertebral endplate that contacts the first support or the second support. And, in some embodiments, the vertically expanding includes introducing a vertical expansion member into the intervertebral space through the single point of entry and into the first support or the second support of the scaffolding to provide a vertical force on adjacent vertebral endplates for the distraction of the intervertebral space.

In some embodiments, the expansion mechanism provides the vertically expanding by expanding the first support or the second support using a means for creating a convex surface that at least substantially complements the concavity of a surface of a vertebral endplate that contacts the first support or the second support. And, in some embodiments, the expansion mechanism provides the vertically expanding by introducing a vertical expansion member into the intervertebral space through the single point of entry and into the first support or the second support of the scaffolding to provide a vertical force on adjacent vertebral endplates for the distraction of the intervertebral space.

It should be appreciated that the vertical expansion member can have several designs, such that the design only need to accomplish the functions taught herein. In some embodiments, the vertical expansion member includes a port for introducing the grafting material after the introducing of the vertical expansion member. In some embodiments, the vertical expansion member is a shim. And, in some embodiments, the vertical expansion member is a shaped shim, such that the vertically expanding includes expanding the first support or the second support in a manner that creates a convex surface that at least substantially complements the concavity of a surface of a vertebral endplate that contacts the first support or the second support.

One of skill will appreciate that the above embodiments are provided for purposes of outlining general concepts, and that several additional embodiments are included in, and can be derived from, the teachings provided herein.

### BRIEF DESCRIPTION OF THE FIGURES

FIGS. 1A-1C illustrate a laterovertically-expandable scaffolding, according to some embodiments.

FIGS. 2A-2C illustrate a method of using a laterovertically-expandable scaffolding, according to some embodiments.

FIGS. 3A-3D illustrate shims that can be used as vertical expansion members, according to some embodiments.

FIG. 4A and 4B illustrate additional vertical expansion mechanisms, according to some embodiments.

### DETAILED DESCRIPTION OF THE INVENTION

The teachings provided herein are generally directed to a method of fusing an intervertebral space in a subject using a laterovertically-expandable scaffolding. For example, the scaffolding can include two elongated segments connected by a hinge, such that the elongated segments can act as support members, or beams, in some embodiments, within an intervertebral disc that has had the nucleus pulposus removed.

The elongated segments can collapse into each other, much like the components of a jackknife collapse into each other, for example, in at least substantially collinear fashion. The elongated segments can rotate such that they cross, and they can connect in such a way that a component for adding a vertical force between vertebrae can be positioned within at least one of the segments. In some embodiments, one of the segments can have a slot that allows for insertion of an expansion mechanism. A shim or other graft material are examples of an expansion mechanism that can expand the distance between the top and bottom walls of at least one of the segments for a distraction of the intervertebral space. If the hinge, for example, is formed by two independent pins operably connecting the two elongated segments in a rotatably articulable relationship, a shim can be introduce into at least one of the elongated segments in a collapsed configuration without the hinge pin obstructing passage of the expansion mechanism. As such, the top and bottom walls of at least one of the segments can be configured or attached in a manner that facilitates the expansion to distract the intervertebral space. An example of such a configuration can include, for example, an arch, a zigzag, or a sinusoidal shape built into at least one of the segments, or a connection between the segments, that facilitates ease of expansion during a distraction.

The elongated segments include designs that support the intervertebral space for a spinal fusion procedure. As such, the segments can be considered as examples of “supports” in some embodiments. Such supports can include any configuration known to one of skill to operate consistent with the teachings provided herein. As such, any at least substantially complementary shapes or forms that will collapse or expand as taught herein can be used. In some embodiments, for example, concentric channels, c-channels, channels and rods, channels and blades, channels, and cylinders, overlapping channels, adjacent beams, adjacent rods, adjacent cylinders, and the like, can all be envisioned as operable in view of the teachings provided herein.

The teachings provided herein are generally directed to a method of fusing an intervertebral space using a laterovertically-expandable scaffolding. The terms “scaffold” and “scaffolding”, for example, can be used interchangeably in some embodiments and can be used to refer to any biocompatible structure or framework, which may be used to provide support as described herein in an intervertebral space in a subject. The term “subject” and “patient” can be used interchangeably in some embodiments and refer to an animal such as a mammal including, but not limited to, non-primates such as, for example, a cow, pig, horse, cat, dog, rat and mouse; and primates such as, for example, a monkey or a human. As such, the terms “subject” and “patient” can also be applied to non-human biologic applications including, but not limited to, veterinary, companion animals, commercial livestock, aquaculture, and the like.

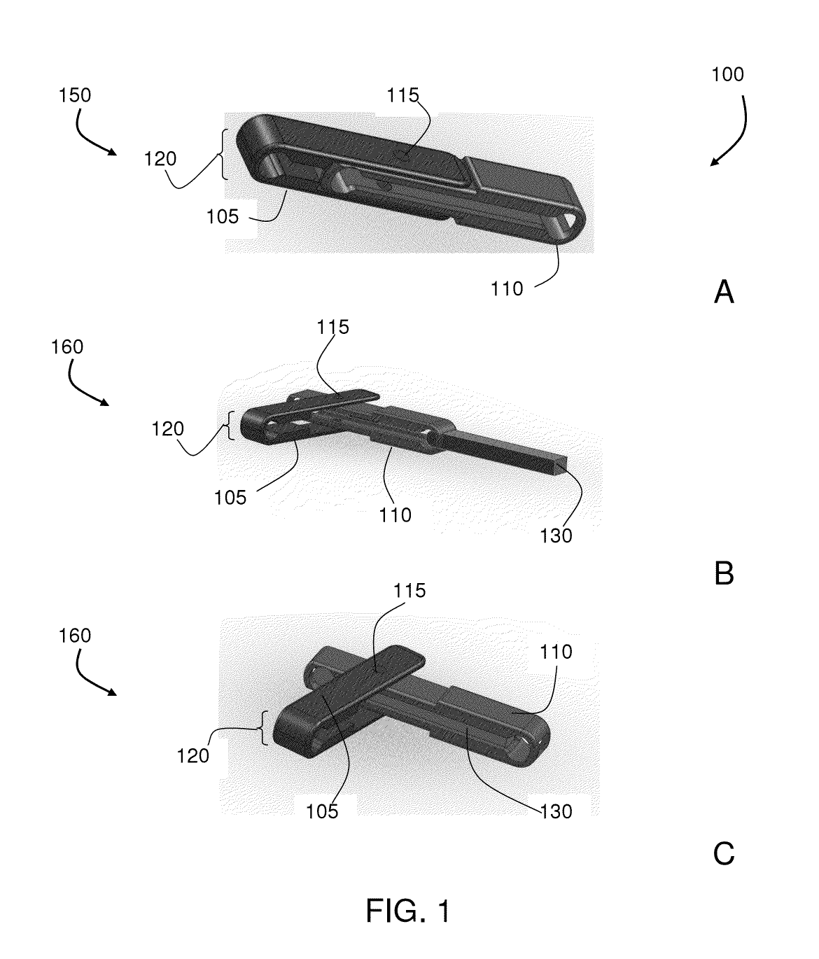

FIGS. 1A-1C illustrate a laterovertically-expandable scaffolding, according to some embodiments. The laterovertically-expandable scaffolding 100 is designed to be operable for supporting an intervertebral disc space. The scaffolding 100 can have at least a first support 105 and a second support 110, the second support 110 operable to laterally collapse into, and laterally expand from, the first support 105 by rotating, or pivoting, at a hinge 115. The laterovertically-expandable scaffolding 100 can be configured to provide a low-profile entry 120 in a collapsed configuration 150 through a single point of entry through the annulus of an intervertebral disc having a intevertebral space created by the removal of the nucleus pulposus from the intervertebral disc. The lateral movement can include a rotation at a point of intersection, the hinge 115, between the first support 105 and the second support 110, such that the lateral movement includes a scissor-like movement between the first support 105 and the second support 110 in the intevertebral space. The collapsed configuration 150 can have the shape of an I for inserting the scaffolding 100 into the intevertebral space, and the expanded configuration 160 can have the shape of an X in the intevertebral space after expansion; and, the intersection is biased by positioning the hinge 115 anteriorly in the intevertebral space to facilitate the adding of a grafting material to the intevertebral space after the expansion of the scaffolding, for example. Moreover, the scaffolding can be configured to be operable with a vertical expansion member 130 that can be inserted into the intevertebral space through the single point of entry and into the first support or the second support of the scaffolding to provide a vertical force on adjacent vertebral endplates for distraction of the intervertebral space. In some embodiments, the vertical expansion member is a shim. In some embodiments, the shim can comprise a non-resorbable polymer material, an inorganic material, a metal, an alloy, or bone.

In some embodiments, the scaffolding comprises at least a first support and a second support, the combination of the first support and the second support operable to laterally expand and vertically expand from a collapsed configuration within an intervertebral space. In these embodiments, the first support and the second support can be at least substantially rigid; and, the first support and the second support can lie at least substantially on the same plane. The collapsed configuration can be configured to provide a low-profile entry through a minimally-invasive single point of entry through the annulus fibrosis of an intervertebral disc, the intervertebral disc having the nucleus pulposus removed. The removal of the nucleus pulposus leaves an intervertebral space for expansion of the laterovertically-expandable scaffolding within the annulus fibrosis using an expansion mechanism for laterally expanding at least a portion of the second support and at least a portion of the first support away from each other; and, vertically expanding at least a portion of the first support or at least a portion of the second support for a distraction of the intervertebral space.

In some embodiments, the expansion mechanism can laterally expand at least a portion of the second support and at least a portion of the first support away from each other, the laterally expanding including a rotation at a point of intersection between the first support and the second support, such that the laterally expanding includes a scissor-like movement between the first support and the second support in the intervertebral space; and, the vertically expanding includes expanding at least a portion of the first support or at least a portion of the second support for a distraction of the intervertebral space, the vertically expanding includes introducing a vertical expansion member into the intervertebral space through the single point of entry and into the first support or the second support of the scaffolding to provide a vertical force on adjacent vertebral endplates for the distraction of the intervertebral space. In these embodiments, the collapsed configuration is configured for the low profile entry through the annulus fibrosis, having the shape of an I for inserting the scaffolding into the intervertebral space through the single point of entry; and, the expanded configuration is configured to provide a stable support for fusing the intervertebral space, having the shape of an X in the intervertebral space, the point of intersection biased anteriorly in the intervertebral space to facilitate the adding of a grafting material and maximize an area of contact between the scaffolding, the grafting material, and the vertebral endplates of the intervertebral space.

It should be appreciated that the collapsed configuration of the scaffolding can be any configuration that will facilitate obtaining the functions taught herein. In some embodiments, the collapsed configuration has the shape of an I for the inserting of the scaffolding into the intervertebral space, and the expanded configuration has the shape of an X in the intervertebral space. In some embodiments, the shape of the X is asymmetrical in the intervertebral space and the intersection is biased anteriorly in the intervertebral space to facilitate the adding of the grafting material and maximize an area of contact between the scaffolding, the grafting material, and the vertebral endplates of the intervertebral space.

The collapsed configuration includes the design of a low profile entry through the annulus fibrosis to allow for a minimally-invasive procedure. In order to facilitate the use of a minimally-invasive procedure, the low profile entry of the collapsed configuration should be a substantially small area of entry having a diameter ranging, for example, from about 5 mm to about 12 mm for the single point of entry through the annulus fibrosis. In some embodiments, the low profile has an area with a diameter ranging from about 2 mm to about 20 mm, from about 3 mm to about 18 mm, from about 4 mm to about 16 mm, from about 5 mm to about 14 mm, from about 6 mm to about 12 mm, from about 7 mm to about 10 mm, or any range therein. In some embodiments, the low profile has an area with a diameter of 2 mm, 4 mm, 6 mm, 8 mm, 10 mm, 12 mm, 14 mm, 16 mm, 18 mm, or any range therein, including any increment of 1 mm in any such diameter or range therein.

It should be appreciated that the lateral and vertical expansions can occur in any manner, using any respective expansion mechanism that will provide the functions of the scaffoldings taught herein. In some embodiments, the laterally expanding includes a rotation at a point of intersection between the first support and the second support, such that the laterally expanding includes a scissor-like movement between the first support and the second support in the intervertebral space. And, in some embodiments, the laterally expanding includes a translation at a point of intersection between the first support and the second support, such that the laterally expanding includes a scissor-like movement in the intervertebral space between the first support and the second support. In some embodiments, the translation occurs without an attachment between the first support and the second support, such that the first support and the second support are translatable relative to one another but without an attachment that otherwise prevents or inhibits their separation. In some embodiments, the first support and the second support can have an attachment, limiting the freedom of movement between the components by at least one degree of freedom. As such, the components are allowed to translate in a limited fashion and are prevented or inhibited from separation.

In some embodiments, the vertically expanding includes expanding the first support or the second support using a means for creating a convex surface that at least substantially complements the concavity of a surface of a vertebral endplate that contacts the first support or the second support. And, in some embodiments, the vertically expanding includes introducing a vertical expansion member into the intervertebral space through the single point of entry and into the first support or the second support of the scaffolding to provide a vertical force on adjacent vertebral endplates for the distraction of the intervertebral space.

It should be appreciated that the vertical expansion member can have several designs, such that the design only need to accomplish the functions taught herein. In some embodiments, the vertical expansion member includes a port for introducing the grafting material after the introducing of the vertical expansion member. In some embodiments, the vertical expansion member is a shim. And, in some embodiments, the vertical expansion member is a shaped shim, such that the vertically expanding includes expanding the first support or the second support in a manner that creates a convex surface that at least substantially complements the concavity of a surface of a vertebral endplate that contacts the first support or the second support. As such, in some embodiments, the scaffoldings provided herein provide an ability to conform to the vertebral endplates in a manner not currently available in the art.

Having the ability to reach such a conformity between the bone and scaffolding merely adds to the improved function that's already provided by the laterovertically expandable scaffoldings taught herein. The scaffoldings taught herein facilitate a maximizing of the contact area around and between the scaffolding, the bone graft material, and the surrounding bone in the intervertebral space. These improvements provide at least an improvement over the state-of-the-art (i) in the initial distraction of the intervertebral space, (ii) stability during fusion; and (iii) bone in-growth during fusion, each of which is highly desired to one of skill in the art.

The positioning of the first component and the second component in the intevertebral space can occur with or without the use of any particular tool. In some embodiments, the positioning, or expansion, can be accomplished by manipulating the scaffolding during it's insertion into the intevertebral space. In some embodiments, the positioning, or expansion, can be accomplished using a particular tool that is configured to manipulate one of the supports relative to the other. For example, a beveled tool can be used to exert a lateral pressure on one of the supports by inserting the beveled tool against a support having a gradually increasing pitch on a complementary bevel that is configured to create the lateral pressure that results in expansion. Such a tool may be referred to as a “pushrod”. In some embodiments, the scaffolding may have a “memory”, such that it wants to expand from it's collapsed state into the desired expanded state after insertion into the intevertebral space. The memory of the scaffolding provides a potential energy for release of the scaffolding into the expanded configuration, the potential energy derived from, for example, a spring steel or other like material that contains such a memory

The laterovertically-expandable scaffolding can comprise any suitable material known to one of skill. One of skill will appreciate that the scaffoldings can have performance characteristics that are near that of a bone structure, in some embodiments, such that the scaffoldings are not too stiff or hard, resulting in a localized loading issue in which the scaffolding puts too much pressure on native bone tissue, and likewise such that the scaffoldings are too flexible or soft, resulting in a localized loading issue in which the bone tissue puts too much pressure on the scaffolding.

Examples of such materials can include non-reinforced polymers, carbon-reinforced polymer composites, PEEK (polyether ketone) and PEEK composites, ULTEM, liquid metal, shape-memory alloys, titanium, titanium alloys, cobalt chrome alloys, stainless steel, ceramics and combinations thereof. A radio-opaque material can be employed to facilitate identifying the location and position of the scaffolding in the spinal disc space. In some embodiments, the scaffolding can comprise a metal frame and cover made of PEEK or ULTEM. Examples of titanium alloys can include alloys of titanium, aluminum, and vanadium, such as Ti₆Al₄V in some embodiments. One of skill can select materials on the basis of desired material performance characteristics. For example, one of skill will look to performance characteristics that can include static compression loading, dynamic compression loading, static torsion loading, dynamic torsion loading, static shear testing, dynamic shear testing, expulsion testing, and subsidence testing. The parameters for upper and lower limits of performance for these characteristics can fall within the range of existing such spinal devices that bear the same or similar environmental conditions during use. For example, a desired static compression loading can be approximately 5000N. A desired dynamic compression loading can have an asymptotic load level of ≧3000N at 5×10⁶ cycles or ≧1500N at 10×10⁶ cycles. The desired load level can range, for example, from about 1.0× to about 2.0×, from about 1.25× to about 1.75×, or any range therein in increments of 0.1×, the vertebral body compression strength. Examples of standard procedures used to test such performance characteristics include ASTM F2077 and ASTM F2624.

Bone ingrowth is desirable in many embodiments. As such, the scaffolding can comprise materials that contain holes or slots to allow for such bone ingrowth. Consistently, the scaffoldings can be coated with hydroxyapatite, or other bone conducting surface, for example, bone morphogenic protein, to facilitate bone ingrowth. Moreover, the surfaces of the scaffoldings can be formed as rough surfaces with protuberances, insets, or projections of any type known to one of skill, such as teeth or pyramids, for example, to grip vertebral endplates, avoid migration of the scaffolding, and encourage engagement with bone ingrowth.

One of skill will appreciate that a variety of surgical methods can be used to implant the scaffoldings taught herein. In some embodiments, the method comprises creating a single point of entry into an intervertebral disc, the intervertebral disc having a nucleus pulposus surrounded by an annulus fibrosis, and the single point of entry is created through the annulus fibrosis. The method includes removing the nucleus pulposus from within the intervertebral disc through the single point of entry, leaving an intervertebral space for expansion of a laterovertically-expandable scaffolding within the annulus fibrosis. The method further includes inserting the laterovertically-expandable scaffolding through the single point of entry into the intervertebral space, the laterovertically-expandable scaffolding having at least a first support and a second support, the combination of the first support and the second support operable to laterally expand and vertically expand from a collapsed configuration within an intervertebral space, such that the laterovertically-expandable scaffolding is configured to provide a low-profile entry in the collapsed configuration through the single point of entry through the annulus. As such, the method further includes expanding the laterovertically-expandable scaffolding.

The expanding can include laterally expanding at least a portion of the second support and at least a portion of the first support away from each other; and, vertically expanding at least a portion of the first support or at least a portion of the second support for a distraction of the intervertebral space. As a method of fusing the intervertebral space, the method further includes adding a grafting material through the single point of entry into the intervertebral space around the laterovertically-expandable scaffolding. In such embodiments, the first support and the second support are each at least substantially rigid; and, the first support and the second support lie at least substantially on the same plane.

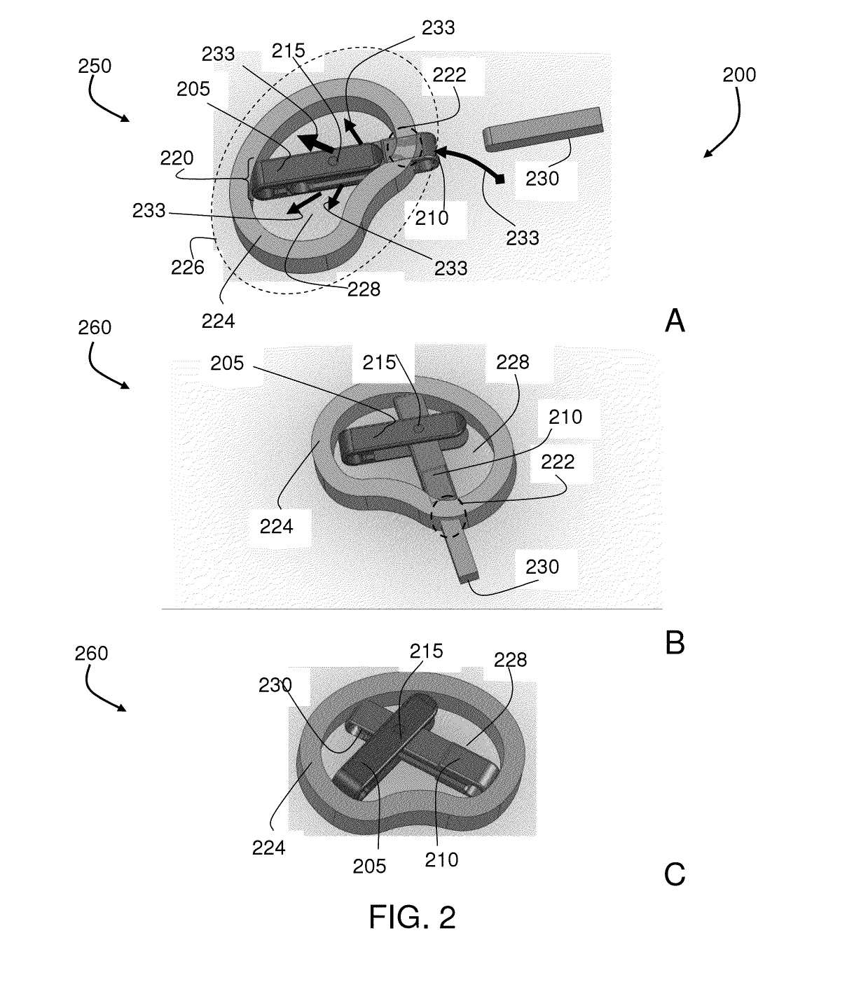

FIGS. 2A-2C illustrate a method of using a laterovertically-expandable scaffolding, according to some embodiments. The laterovertically-expandable scaffolding 200 is designed to be operable for supporting an intervertebral disc space. The scaffolding 200 can have at least a first support 205 and a second support 210, the second support 210 operable to laterally collapse into, and laterally expand from, the first support 205 by rotating, or pivoting, at a hinge 215. The laterovertically-expandable scaffolding 200 can be configured to provide a low-profile entry 220 in a collapsed configuration 250 through a single point of entry 222 through the annulus fibrosis 224 of an intervertebral disc 226 having a intevertebral space 228 created by the removal of the nucleus pulposus (not shown) from the intervertebral disc 226. The lateral expansion can include a rotation at a point of intersection, the hinge 215, between the first support 205 and the second support 210, such that the lateral expansion includes a scissor-like movement between the first support 205 and the second support 210 in the intevertebral space 228.

It should be appreciated that the single point of entry can be made in any manner that will facilitate obtaining the functions taught herein. In some embodiments, the single point of entry through the annulus fibrosis is configured to accommodate the low profile having an area having an effective diameter ranging from about 5 mm to about 12 mm. In some embodiments, the low profile has an area with a diameter ranging from about 2 mm to about 20 mm, from about 3 mm to about 18 mm, from about 4 mm to about 16 mm, from about 5 mm to about 14 mm, from about 6 mm to about 12 mm, from about 7 mm to about 10 mm, or any range therein. In some embodiments, the low profile has an area with a diameter of 2 mm, 4 mm, 6 mm, 8 mm, 10 mm, 12 mm, 14 mm, 16 mm, 18 mm, or any range therein, including any increment of 1 mm in any such diameter or range therein.

The collapsed configuration 250 can have the shape of an I for inserting the scaffolding 200 into the intevertebral space 228, and the expanded configuration 260 can have the shape of an X in the intevertebral space 228 after expansion; and, the intersection is biased by positioning the hinge 215 anteriorly in the intevertebral space 228 to facilitate the adding of a grafting material 233 to the intevertebral space 228 after the expansion of the scaffolding 200, for example. Moreover, one of skill will appreciate having the ability to include a vertical expansion member in the intevertebral space 228. As such, in some embodiments, the method comprises introducing a vertical expansion member 230 into the intevertebral space through the single point of entry and into the first support or the second support of the scaffolding to provide a vertical force on adjacent vertebral endplates for distraction of the intervertebral space. In some embodiments, the vertical expansion member is a shim. As described herein, shims, shaped shims, and any of a variety of means for vertically expanding the scaffolding can be used with the teachings set-forth herein. The grafting material 233 can be added in the same port, or a different port, as the vertical expansion member.

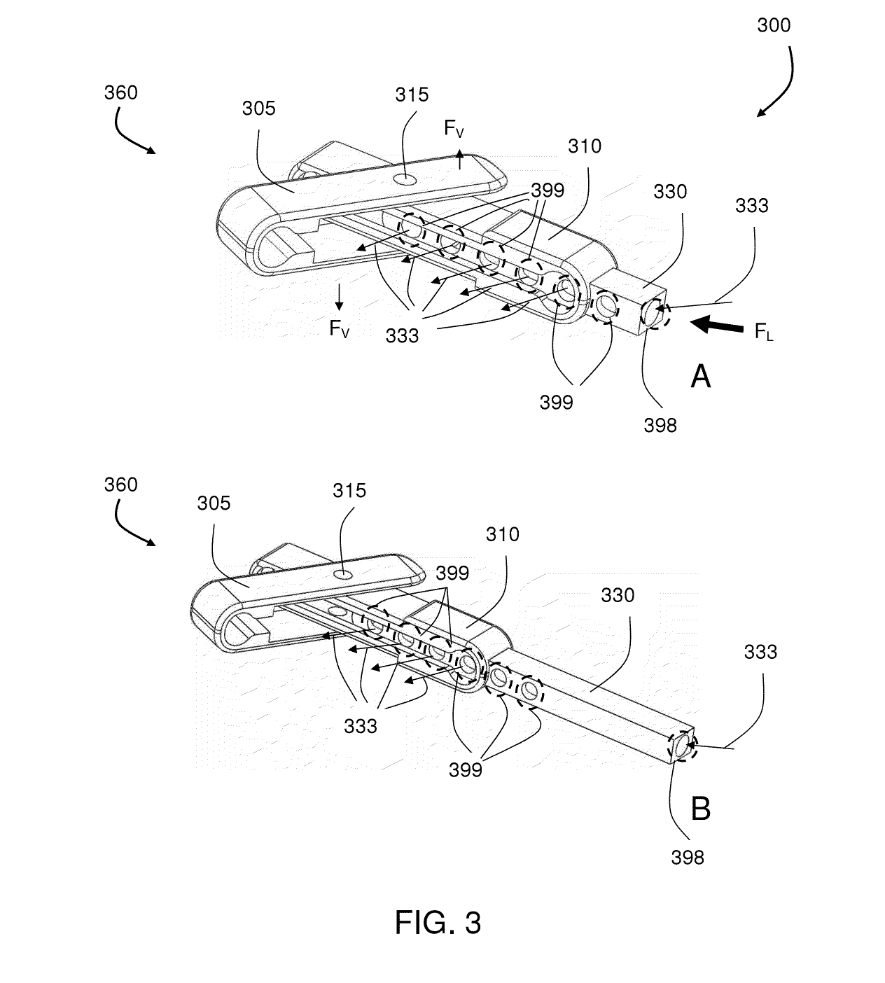

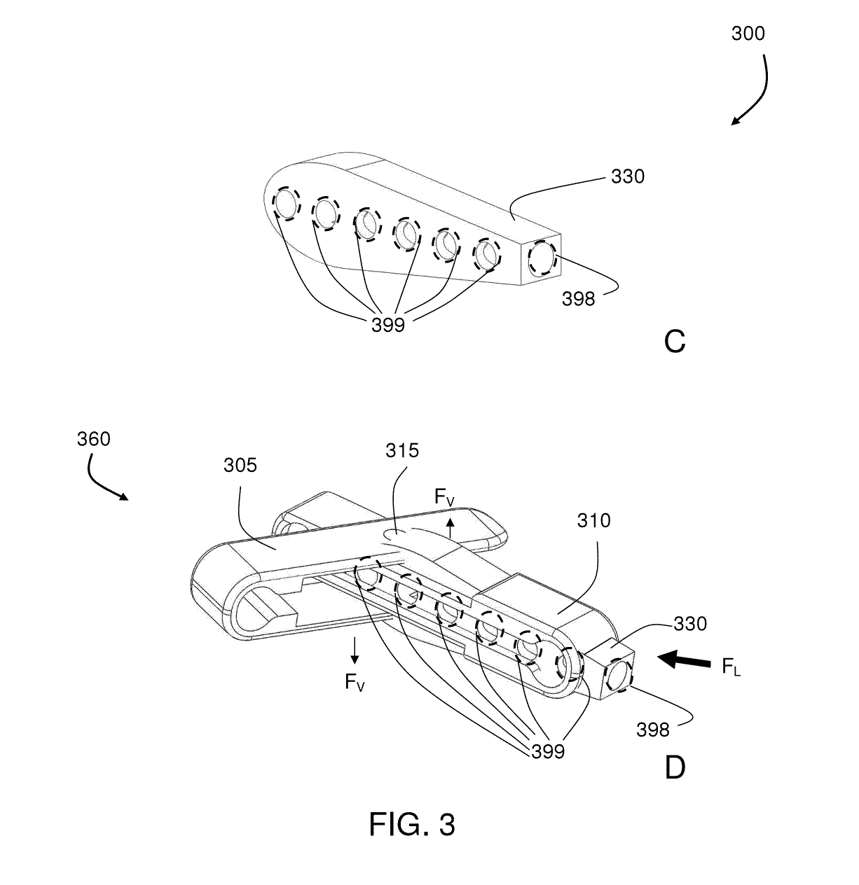

FIGS. 3A-3D illustrate shims that can be used as vertical expansion members, according to some embodiments. The shims can have a variety of shapes, and the shapes can be designed to complement or otherwise function with the scaffolding. In some embodiments, the shim can have the shape of a wedge, and the shape can include protuberances such as serrations to improve a friction fit between the scaffolding and the shim. In some embodiments, the material selections of the scaffolding and shim can provide a substantial friction fit. In some embodiments, the shim can have an elliptical shape to create a convex surface on the scaffolding for mating with a concave surface on a vertebral endplate. In some embodiments, the shim can be rectangular in cross-section, such that the shim is inserted into the scaffolding using it's thinnest dimension and rotated after entry to obtain the desired distraction between the opposing vertebral endplates. In some embodiments, the shim is elliptical on at least one side of the shim and flat on at least two opposing sides of the shim. The shim is inserted such that the two opposing flat sides represent the thinnest dimension and rotated after entry to obtain the desired distraction, the distraction including the creation of a convex surface on the scaffolding that contact a concave surface on an endplate. In some embodiments, the rotated shims can be designed to lock in place, and sometimes reversibly, by interlocking the shim with a groove or other mating surface designed to hold the shim in a desired orientation upon the rotation. In some embodiments, the shims can be designed to obtain a desired orientation between the vertebrae that form the intervertebral space. For example, a shim can be used to create or induce a “pitch” between the vertebrae to achieve a therapeutic effect such as, for example, a modified distraction that further opens foramina or releases pressure on the nerve and facets without proportionally inducing as much distraction anteriorly.

FIG. 3A illustrates an expansion shim 330 that is intended as a permanent placement in the scaffolding 300, and FIG. 3B shows an expansion shim 330 as placed in the scaffolding 300. As shown in FIG. 3A, a lateral force, F_{L}, is used to place the expansion shim 330 in the scaffolding 300. A vertical force, F_{V}, is created through the placement of the expansion shim 330 into the scaffolding 300 to cause a distraction of an invertebral space. FIG. 3C illustrates the elliptical type of expansion shim 330, and FIG. 3D show the elliptical shim 330 as placed in the scaffolding 300. As shown in FIG. 3A, a lateral force, F_{L}, is used to place the elliptical shim 330 in the scaffolding 300. A vertical force, F_{V}, is created through the elliptical shim 330 to cause a distraction of an invertebral space. In these embodiments, the placement of the shim 330 is shown in an expanded configuration 360 of the scaffolding 300. The scaffolding 300 can have at least a first support 305 and a second support 310, the second support 310 operable to laterally collapse into, and laterally expand from, the first support 305 by rotating, or pivoting, at a hinge 315. As shown in FIGS. 3A-3D, the shims 330 have an entry port 398 for adding graft material 333, and at least one exit port 399 for distribution of the graft material 333 into the intervertebral space. FIGS. 3A, 3C, and 3D can be used in a permanent placement of the shim 330, whereas FIG. 3B shows a “trial” shim 330, which is temporarily inserted for the introduction of the graft material 333, removed, and then a permanent shim is placed.

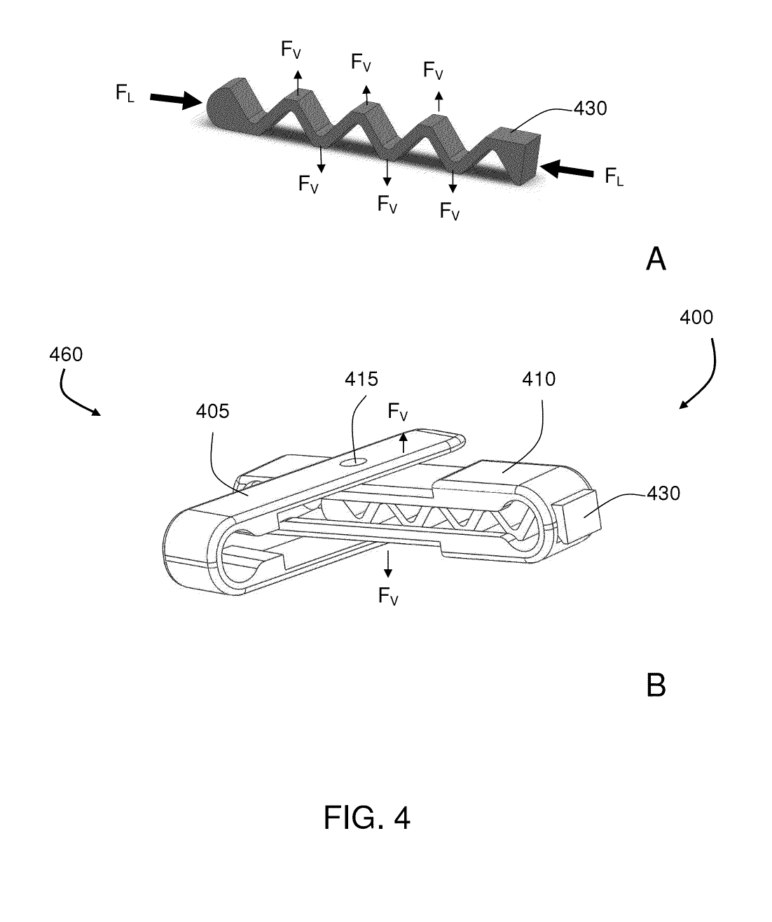

FIG. 4A and 4B illustrate an additional vertical expansion mechanism, according to some embodiments. One of skill will appreciate that a variety of mechanisms can be used to obtain a desired amount and type of distraction. In some embodiments, a coil mechanism (not shown) can be used, wherein the coil in an axially expanded state has a smaller diameter than the coil in an axially compressed state, and a compression of the coil can create a desired amount of distraction. In some embodiments, the concept of the wall anchor can be used, where a cylinder having linear cuts is compressed, and portions of the cylinder expand outward to achieve a desired amount of distraction. In some embodiments, the mechanism of the scissor jack can be used, where the shim is designed having a scissor-jack type mechanism that can be expanded to achieve a desired amount of distraction. Likewise, other such expansive mechanisms can be used, such as the sinusoidal configurations commonly used on stents, in which an expansion of such a sinusoidally compressed structure can create a desired amount of distraction. FIG. 4A illustrates the sinusoidal type of expansion shim 430, and FIG. 4B shows the sinusoidal shim 430 as placed in the scaffolding 400. As shown in FIG. 4A, a lateral force, F_{L}, is used to place and compress the sinusoidal shim 430 in the scaffolding 400. Upon compression due to F_{L}, a vertical force, F_{V}, is created through the sinusoidal shim 430 to cause a distraction of an invertebral space. The placement of the sinusoidal shim 430 is shown in an expanded configuration 460 of the scaffolding 400. The scaffolding 400 can have at least a first support 405 and a second support 410, the second support 410 operable to laterally collapse into, and laterally expand from, the first support 405 by rotating, or pivoting, at a hinge 415.

The methods and systems provided herein include the use of bone graft materials known to one of skill. Materials which may be placed or injected into the intevertebral space include solid or semi-solid grafting materials, bone from removed from patient's facet, an iliac crest harvest from the patient, and bone graft extenders such as hydroxyapatite, demineralized bone matrix, and bone morphogenic protein. Examples of solid or semi-solid grafting material components include solid fibrous collagen or other suitable hard hydrophilic biocompatible material. Some materials may also include swelling for further vertical expansion of the intervertebral disc space.

The scaffolding systems taught herein can be provided to the art in the form of kits. A kit can contain, for example, a scaffolding, a vertical expansion member, and a bone graft material. In some embodiments, the kit will contain an instruction for use. The vertical expansion member can be any vertical expansion mechanism or means taught herein. For example, the vertical expansion member can be a shim. In some embodiments, the kit includes a graft-injection shim for temporarily distracting the intervertebral space, the graft-injection shim having a port for receiving and distributing the bone graft material in the intervertebral space. In these embodiments, the graft-injection shim can remain as a permanent shim or be removed and replaced with a permanent shim.

One of skill will appreciate that the teachings provided herein are directed to basic concepts that can extend beyond any particular embodiment, embodiments, figure, or figures. It should be appreciated that any examples are for purposes of illustration and are not to be construed as otherwise limiting to the teachings.

We claim:

1. A method of fusing an intervertebral space using a laterovertically-expandable scaffolding, the method comprising:
creating a single point of entry into an intervertebral disc, the intervertebral disc having a nucleus pulposus surrounded by an annulus fibrosis, and the single point of entry is created through the annulus fibrosis;
removing the nucleus pulposus from within the intervertebral disc through the single point of entry, leaving an intervertebral space for expansion of a laterovertically-expandable scaffolding within the annulus fibrosis;
inserting the laterovertically-expandable scaffolding through the single point of entry into the intervertebral space, the laterovertically-expandable scaffolding having at least a first support and a second support, the combination of the first support and the second support operable to laterally expand and vertically expand from a collapsed configuration within an intervertebral space, such that the laterovertically-expandable scaffolding is configured to provide a low-profile entry in the collapsed configuration through the single point of entry through the annulus;
expanding the laterovertically-expandable scaffolding, the expanding including
laterally expanding at least a portion of the second support and at least a portion of the first support away from each other; and,
vertically expanding at least a portion of the first support or at least a portion of the second support for a distraction of the intervertebral space;
and,
adding a grafting material to the intervertebral space through the single point of entry into the intervertebral space around the laterovertically-expandable scaffolding;
wherein,
the first support and the second support are each at least substantially rigid; and,
the first support and the second support lie at least substantially on the same plane.

2. The method of claim 1, wherein, the laterally expanding includes a rotation at a point of intersection between the first support and the second support, such that the lateral movement includes a scissor-like movement between the first support and the second support in the intervertebral space.

3. The method of claim 1, wherein, the laterally expanding includes a translation at a point of intersection between the first support and the second support, such that the lateral movement includes a scissor-like movement in the intervertebral space between the first support and the second support.

4. The method of claim 1, wherein the inserting includes using the collapsed configuration in the shape of an I during the inserting of the scaffolding into the intervertebral space, and using the expanded configuration in the shape of an X in the intervertebral space.

5. The method of claim 1, wherein the inserting includes configuring the low profile entry to have an area with an effective diameter ranging from about 5 mm to about 12 mm for a minimally-invasive single point of entry through the annulus fibrosis.

6. The method of claim 4, wherein the expanding includes configuring the scaffolding into an asymmetrical X in the intervertebral space, the configuring including biasing the intersection anteriorly in the intervertebral space to facilitate the adding of the grafting material and maximize an area of contact between the scaffolding, the grafting material, and the vertebral endplates of the intervertebral space.

7. The method of claim 1, wherein the vertically expanding includes introducing a vertical expansion member into the intervertebral space through the single point of entry and into the first support or the second support of the scaffolding to provide a vertical force on adjacent vertebral endplates for the distraction of the intervertebral space.

8. The method of claim 7, further comprising introducing the grafting material through a port in the vertical expansion member after the introducing of the vertical expansion member.

9. The method of claim 7, wherein the vertical expansion member is a shaped shim, such that the vertically expanding includes expanding the first support or the second support in a manner that creates a convex surface that at least substantially complements the concavity of a surface of a vertebral endplate that contacts the first support or the second support.

10. A laterovertically expandable scaffolding for fusing an intervertebral disc space, the scaffolding comprising:
at least a first support and a second support, the combination of the first support and the second support operable to laterally expand and vertically expand from a collapsed configuration within an intervertebral space;
wherein,
the first support and the second support are at least substantially rigid;
the first support and the second support lie at least substantially on the same plane; and, the collapsed configuration is configured to provide a low-profile entry through a minimally-invasive single point of entry through the annulus fibrosis of an intervertebral disc, the intervertebral disc having the nucleus pulposus removed, leaving an intervertebral space for expansion of the laterovertically-expandable scaffolding within the annulus fibrosis using an expansion mechanism for
laterally expanding at least a portion of the second support and at least a portion of the first support away from each other; and,
vertically expanding at least a portion of the first support or at least a portion of the second support for a distraction of the intervertebral space.

11. The scaffolding of claim 10, wherein the collapsed configuration has the shape of an I for inserting the scaffolding into the intervertebral space, and the expanded configuration has the shape of an X in the intervertebral space.

12. The scaffolding of claim 11, wherein the shape of the X is asymmetrical in the intervertebral space, and the intersection is biased anteriorly in the intervertebral space to facilitate the adding of the grafting material and maximize an area of contact between the scaffolding, the grafting material, and the vertebral endplates of the intervertebral space.

13. The scaffolding of claim 10, wherein the low profile entry has an area with an effective diameter ranging from about 5 mm to about 12 mm for a minimally-invasive single point of entry through the annulus fibrosis.

14. The scaffolding of claim 10, wherein, the expansion mechanism provides the laterally expanding through a rotation at a point of intersection between the first support and the second support, such that the lateral movement includes a scissor-like movement between the first support and the second support in the intervertebral space.

15. The scaffolding of claim 10, wherein, the expansion mechanism provides the laterally expanding through a translation at a point of intersection between the first support and the second support in the intervertebral space, such that the lateral movement includes a scissor-like movement between the first support and the second support.

16. The scaffolding of claim 10, wherein the expansion mechanism provides the vertically expanding by introducing a vertical expansion member into the intervertebral space through the single point of entry and into the first support or the second support of the scaffolding to provide a vertical force on adjacent vertebral endplates for the distraction of the intervertebral space.

17. The scaffolding of claim 16, wherein the vertical expansion member includes a port for introducing the grafting material after the introducing of the vertical expansion member.

18. The scaffolding of claim 16, wherein the vertical expansion member is a shaped shim, such that the vertically expanding includes expanding the first support or the second support in a manner that creates a convex surface that at least substantially complements the concavity of a surface of a vertebral endplate that contacts the first support or the second support

19. A laterally expandable scaffolding for fusing an intervertebral disc space, the scaffolding comprising:
at least a first support and a second support, the combination of the first support and the second support operable to laterally expand and vertically expand from a collapsed configuration within an intervertebral space; and,
an expansion mechanism;
wherein,
the first support and the second support are at least substantially rigid;
the first support and the second support lie at least substantially on the same plane;
the collapsed configuration is configured to provide a low-profile entry through a minimally-invasive single point of entry through the annulus fibrosis of an intervertebral disc, the intervertebral disc having the nucleus pulposus removed, leaving an intervertebral space for expansion of the laterovertically-expandable scaffolding within the annulus fibrosis using, the expansion mechanism having a means for
laterally expanding at least a portion of the second support and at least a portion of the first support away from each other, the laterally expanding includes a rotation at a point of intersection between the first support and the second support, such that the laterally expanding includes a scissor-like movement between the first support and the second support in the intervertebral space; and,
vertically expanding at least a portion of the first support or at least a portion of the second support for a distraction of the intervertebral space, the vertically expanding includes introducing a vertical expansion member into the intervertebral space through the single point of entry and into the first support or the second support of the scaffolding to provide a vertical force on adjacent vertebral endplates for the distraction of the intervertebral space;
and,
the collapsed configuration is configured for the low profile entry through the annulus fibrosis, having the shape of an I for inserting the scaffolding into the intervertebral space through the single point of entry; and,
the expanded configuration is configured to provide a stable support for fusing the intervertebral space, having the shape of an X in the intervertebral space, the point of intersection biased anteriorly in the intervertebral space to facilitate the adding of a grafting material and maximize an area of contact between the scaffolding, the grafting material, and the vertebral endplates of the intervertebral space.

20. The scaffolding of claim 19, wherein the low profile entry has an area with an effective diameter ranging from about 5 mm to about 12 mm for a minimally-invasive single point of entry through the annulus fibrosis.

21. The scaffolding of claim 19, wherein the means for the laterally expanding includes a rotation at the point of intersection between the first support and the second support, such that the lateral movement includes a scissor-like movement between the first support and the second support in the intervertebral space.

22. The scaffolding of claim 19, wherein the means for the laterally expanding includes a translation at the point of intersection between the first support and the second support in the intervertebral space, such that the lateral movement includes a scissor-like movement between the first support and the second support.

23. The scaffolding of claim 19, wherein the vertical expansion member includes a port for introducing the grafting material after the introducing of the vertical expansion member.

24. The scaffolding of claim 19, wherein the vertical expansion member is a shaped shim, such that the vertically expanding includes expanding the first support or the second support in a manner that creates a convex surface that at least substantially complements the concavity of a surface of a vertebral endplate that contacts the first support or the second support.
